(19) United States
(12) Patent Application Publication
(10) Pub. No.: US 2009/0226906 A1
(43) Pub. Date: Sep. 10, 2009

(54) METHODS AND COMPOSITIONS FOR REDUCING NUCLEOTIDE IMPURITIES

(75) Inventors: David Z. Xie, Allston, MA (US); Philip R. Buzby, Brockton, MA (US); Timothy D. Harris, Toms River, NJ (US)

Correspondence Address: COOLEY GODWARD KRONISH LLP;ATTN: Patent Group, Suite 1100, 777 - 6th Street, NW, WASHINGTON, DC, 20001, US

(73) Assignee: HELICOS BIOSCIENCES CORPORATION, Cambridge, MA (US)

(21) Appl. No.: 12/113,501

(22) Filed: May 1, 2008

Related U.S. Application Data
(63) Continuation-in-part of application No. 12/043,033, filed on Mar. 5, 2008, now abandoned.

(51) Int. Cl. C12Q 1/68; C12N 9/00; C12P 19/34

(52) U.S. Cl. 435/6; 435/183; 435/91.2

(57) ABSTRACT

The disclosure provides methods and compositions for reducing nucleotide impurities in reagents and reaction mixtures. Generally, the methods of the invention involve the inclusion of so-called “scrubbing oligonucleotides” (or “scrubbers”) that preferentially incorporate nucleotide impurities, thereby reducing available free impurities. The disclosure further provides methods of sequencing a target nucleic acid by synthesis that utilize “live” scrubbing. Scrubbing oligonucleotides of various structures are disclosed, including hairpin scrubbers and homopolymeric scrubbers.

### TECHNICAL FIELD

The invention is in the field of molecular biology and, more specifically, pertains to methods of reducing nucleotide impurities in reagents used for nucleic acid synthesis and analysis.

### BACKGROUND OF THE INVENTION

A number of initiatives are currently underway to obtain sequence information directly from millions of individual molecules of DNA in parallel. The real-time single molecule sequencing-by-synthesis technologies rely on the detection of fluorescent nucleotides as they are incorporated into a nascent strand of DNA that is complementary to the template being sequenced. An example of asynchronous single molecule sequencing by synthesis is illustrated in FIG. 1. As illustrated, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers are a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-sequencing technologies also exist.

One shortcoming of single molecule sequencing is its sensitivity to impurities and the resulting relatively high per-read error rate. The process of single molecule DNA sequencing is sensitive to a wide variety of impurities arising from numerous potential sources. The errors may arise from the incorporation of properly matching nucleotides (with or without a functional fluorescent dye), as well as from the incorporation of mismatching nucleotides. For instance, the incorporation of a “dark” nucleotide (i.e., either a natural unmodified dNTP without a functional fluorescent dye or a modified dNTP without a functional fluorescent dye) will produce a false deletion in the sequence read. Even traces of cross-contamination of a dye labeled dNTP into a second dye dNTP, e.g., dye-dCTP contamination in dye-dATP gives rise to potential substitution errors. Any source of materials (e.g., polymerase, other enzymes, buffers, or labeled nucleotides themselves) might add exogenous nucleotides that do not contain a label, thus contributing to the errors. Additionally, the impurities may result from the breakdown of dye-labeled nucleotides, which produces nucleotides without a functional label but are capable of outcompeting their labeled analogs. Although pre-purification of reagents by HPLC or other standard purification methods may be used to remove pre-existing impurities, this step does not avoid the contaminants produced as a result of the continuous degradation of labeled nucleotides during storage or during the sequencing reaction.

One solution to the problem utilizes a “pre-scrubbing” system, in which each nucleotide solution is pre-treated to remove potentially contaminating nucleotides. To this end, each nucleotide solution is reacted prior to use with immobilized DNA complementary to each of the possibly contaminating nucleotides of other species. For example, a dATP solution is allowed to react with immobilized poly(dA), poly(dG), or poly(dC), with appropriate primers and polymerase, so as to incorporate any contaminating dTTP, dCTP and dGTP nucleotides into the DNA. However, this “pre-scrubbing” system does not reduce impurities that arise due to the nucleotide degradation during sequencing, nor does it remove the “dark” nucleotides of the same species (in the example above, the dark dATP is not removed from the solution of dye-labeled dATP).

Another solution, described in US Pat. App. Pubs. Nos. 2006/0172313 and 2006/0263790, is aimed at reducing misincorporation events by adding into the reaction mixture nucleotide derivatives that are recognized by the polymerase, and will temporarily occupy the incorporation site, however, they are unable to form a 3′-5′ covalent bond. For instance, three species of the unincorporating derivatives (C, G, and T) may be added to the labeled fourth species (A), thereby reducing incorporation of C, G, and T impurities. Similarly to the “pre-scrubbing” system described above, while this method reduces incorporation of mismatching nucleotides, it does not remove the matching dark nucleotides from the reaction mixture.

Accordingly, there is a need for methods that improve fidelity of the sequencing reactions, particularly, methods that reduce nucleotide impurities in various reagents and reaction mixtures.

### SUMMARY OF THE INVENTION

The invention provides methods and compositions for reducing nucleotide impurities in reagents and reaction mixtures. Generally, the methods of the invention involve the inclusion of so-called “scrubbing oligonucleotides” (or “scrubbers”) that allow polymerase to preferentially incorporate nucleotide impurities into such scrubbers, thereby reducing available free impurities. The methods of the invention may be particularly useful for reducing the amount of nucleotide impurities in systems that are highly sensitive to such impurities, such as, for example, in single molecule sequencing by synthesis. Scrubbing oligonucleotides of the invention can be used, for example, to purify optically labeled nucleotides and other reagents and reaction mixtures as described below. In the illustrative embodiments, the nucleotides are fluorescently labeled deoxyribonucleotides.

This invention is based, at least in part, on the realization that, despite standard purification, polymerases may contain residual amounts of contaminant nucleotides carried over from the source. Thus, in some embodiments, a scrubbing oligonucleotide is added to a polymerase under conditions allowing at least some residual nucleotides, if present, to be incorporated in the scrubber. Accordingly, the invention provides a composition comprising at least one type of a scrubbing oligonucleotide in solution and a polymerase. In further related embodiments, the compositions additionally contain one or more labeled nucleotides to be purified (e.g., optically labeled nucleotides). In some embodiments, only one species of labeled nucleotide is present in the mixture along with at least one scrubbing oligonucleotide that is designed to remove the nucleotide impurities of the same species as the labeled nucleotide.

The invention is further based, at least in part, on the realization that certain labeled nucleotides may degrade during their handling and even in the course of their intended use, for example, during a sequencing-by-synthesis process. Accordingly, in some embodiments of the invention, the scrubbing oligonucleotides are added “live” while a target nucleic acid is undergoing polymerization (e.g., the target nucleic is being sequenced by synthesis).

Scrubbing oligonucleotides of the invention comprise a double-stranded region, an optional loop region (in the case of a hairpin structure), and a single-stranded overhang region that allows for incorporation of one or more nucleotides at the 3′ end of the double-stranded region. In some embodiments, the incorporation site contains a homopolymeric sequence (e.g., TTTTT, as illustrated in FIG. 3C).

The invention further provides methods of sequencing a target nucleic acid by synthesis that utilize the “live” scrubbing as described here. Generally, in such methods, the target nucleic acid is exposed to a) polymerase, b) at least one type of a labeled nucleotide (e.g., A, G, T, U, or C), and c) a scrubbing oligonucleotide, under conditions that allow the polymerase to incorporate the labeled nucleotide(s) into the chain complementary to the target nucleic acid. Thereupon, the sequence of the target nucleic acid is determined based upon the order of incorporation of the labeled nucleotide(s) into the complementary chain. In the illustrative embodiments, the sequencing is performed at a single molecule level, more specifically, wherein multiple target nucleic acid molecules are anchored to a solid support and are individually optically resolvable.

### BRIEF DESCRIPTION OF THE FIGURES

FIGS. 1A and 1B illustrate a typical process of single molecule sequencing by synthesis. 1) “Capture probes” (T(50) oligonucleotides also functioning as primers) are covalently bound with “5′ down” to a surface. 2) Genomic DNA is fragmented, and a polyA tail and a Cy3 label are added at 3′of each fragment. These DNA templates are then hybridized to the capture probes. 3) The captured templates are imaged to establish their location. 4) The captured templates are incubated with a Cy5-labeled nucleotide and a polymerase mixture to allow the polymerization reaction to proceed. 5) The surface is rinsed to wash out unincorporated nucleotides and other reagents. 6) The incorporated nucleotides are imaged and associated with each template by their location. 7) The Cy5 label is chemically cleaved off. 8) The process is repeated with another type of nucleotide.

FIG. 2 shows results of a study comparing missing-base error rates in a sequencing-by-synthesis process as described in the Examples: 1) nucleotides were purified by HPLC and pre-scubbed; 2) nucleotides were purified by HPLC only; 3) nucleotides were purified by HPLC, and live scrubbing was performed during the sequencing reaction.

FIG. 3 illustrates a primary sequence of a general hairpin structure (FIG. 3A) and two specific examples of hairpin scrubbers used in the Examples (only an adenine scrubber is shown; the scrubbing site is underlined). FIG. 3B shows a single-site nucleotide scrubber; FIG. 3B shows a multi-site, homopolymeric, adenine scrubber.

FIG. 4 shows results of several studies comparing the total error rates (missing bases, insertions, and substitutions) in sequencing by synthesis, utilizing single-site scrubbers (bars 1-6) and homopolymeric scrubbers (bar 7).

FIGS. 5A and 5B provide chemical structures for certain labeled nucleotides used in the Examples.

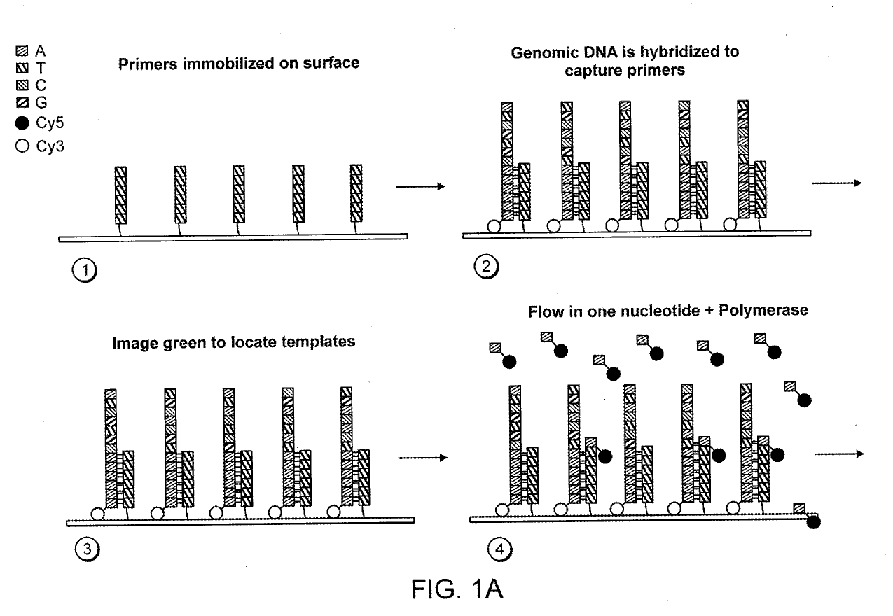

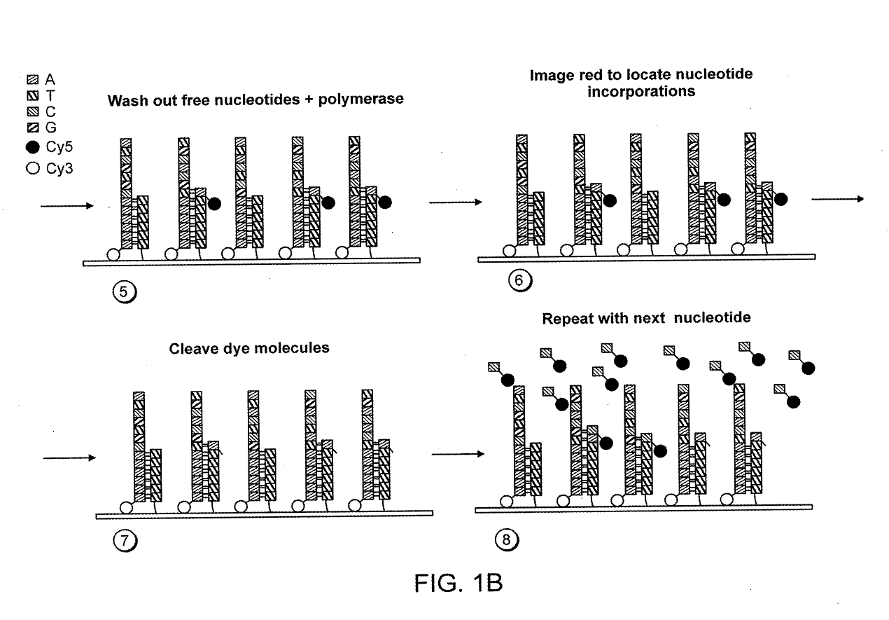

### DETAILED DESCRIPTION OF THE INVENTION

The invention provides methods and compositions for reducing nucleotide impurities in reagents and reaction mixtures. Generally, the methods of the invention involve the inclusion of so-called “scrubbing oligonucleotides” (or “scrubbers”) that allow a polymerase to preferentially incorporate impurities into such scrubbers, thereby reducing available free impurities.

### Scrubbing Oligonucleotides

In accordance with the invention, scrubbing oligonucleotides comprise a double-stranded region, an optional loop (in the case of a hairpin structure), and a single-stranded 5′ overhang region that allows for incorporation of one or more nucleotides at the 3′ end of the double-stranded stem region. In the case of non-hairpin structures, both ends of the double-stranded region may have overhangs.

The length of the double-stranded region may vary. In general, it should be of sufficient length to serve as a substrate for polymerase and to provide a relatively stable structure. For example, the double-stranded region may be 10-100, 10-75, 10-50, 15-50, 15-35, 15-25, or about 20 bps long. In preferred embodiments, the double-stranded region has a GC content of above 40%, above 45%, above 50%, or above 60%. Accordingly, scrubbing oligonucleotides with higher melting temperatures may be preferred. In some embodiments, a scrubbing oligonucleotide has a melting temperature higher than: 65, 67, 70, 72, 75, 77, or 80° C.

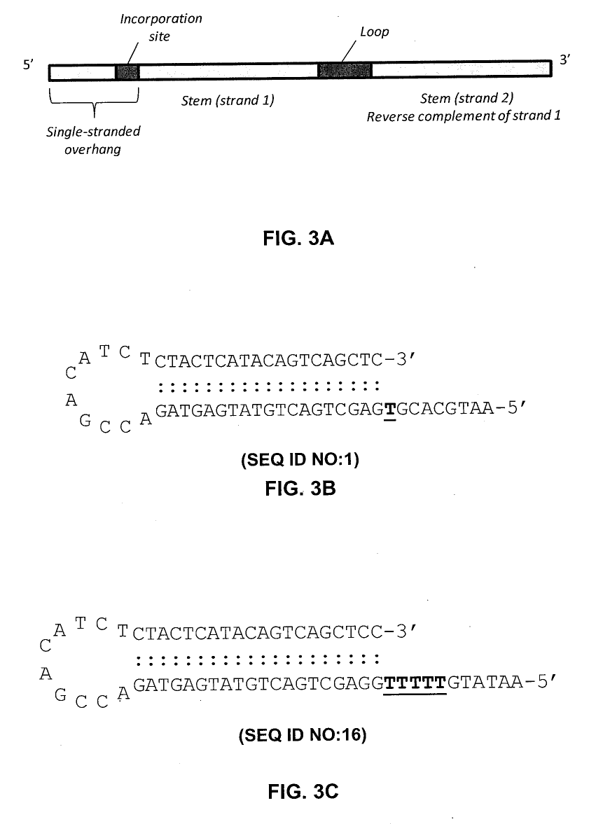

The length of the single-stranded 5′ overhang may also vary. For example, the length of the single-stranded region may be 1-50, 1-35, 1-20, 5-15, or about 10 nts long. The scrubbers are designed to be capable of incorporating one or more contaminant nucleotides in one or more of the first N position(s) most proximal to a 3′ end (incorporation site), wherein N is 7, 6, 5, 4, 3, 2, or 1. Unless otherwise specified, scrubbing oligonucleotides are designated according to the nucleotide species which they will incorporate at the position immediate to the 3′ end, such as A-scrubber (see, e.g., FIGS. 3B and 3C), T/U-scrubber, G-scrubber, and C-scrubber.

In some embodiments, the incorporation site contains as a homopolymeric sequence consisting of 7, 6, 5, 4, 3, or 2 identical bases in a row (e.g., “TTTTT” as in the case of a homopolymeric A-scrubber illustrated in FIG. 3C).

In some embodiments, the scrubbing nucleotides may have a hairpin structure such as illustrated in FIG. 3A, with specific exemplary embodiments shown in FIGS. 3B and 3C. The length of the loop region may vary, and may be for example, 5-30, 5-20, 5-15, or about 10 nts. In the illustrative embodiments provided in the Examples, scrubbers have sequences as set out in SEQ ID NOs:1-4.

In preferred embodiments, the scrubbers are used in solution, while in other embodiments, the scrubbers may be bound to a support.

### Scrubbing Methods

Scrubbing oligonucleotides of the invention can be used to purify labeled nucleotides and other reagents and reaction mixtures. Any such reagent or mixture may be pre-purified by HPLC or other standard methods, as well as subjected to additional purification techniques following, or concurrently with, scrubbing. It may be advantageous to have scrubbers bound to a solid support to facilitate removal of impurity-loaded scrubbers from the solution. For example, in some embodiments, one or more scrubbing oligonucleotides are added to a polymerase solution under conditions allowing residual nucleotide impurities to be incorporated in the scrubbers. In other embodiments, one or more scrubbing oligonucleotides are used for removing contaminant nucleotides from a nucleotide preparation. For example, fluorescently labeled nucleotides may degrade during storage, resulting in contaminating “dark” nucleotides. In such embodiments, in addition to the scrubbers, a polymerase is added to the solution of labeled nucleotide(s) to mediate incorporation of impurities in the scrubbers. For example, a labeled nucleotide (A, G, T, U, or C) may be “scrubbed” by adding a scrubber that incorporates nucleotides of the same species (A, G, T, U, or C, respectively).

In other embodiments, the scrubbing is performed “live” during the intended use of the reagents. Generally, in such methods, a target nucleic acid is exposed to a) polymerase, b) at least one type of a labeled nucleotide (e.g., A, G, T, U, or C), and c) a scrubbing oligonucleotide, under conditions that allow the polymerase to incorporate the labeled nucleotide(s) into the chain complementary to the target nucleic acid, while the nucleotide impurities, if present, are incorporated in the scrubbers.

Thus, the invention provides compositions that comprise at least one type of a scrubbing oligonucleotide in solution and at least one of the following:
a) at least one type of labeled nucleotide species chosen from A, G, T, U, or C;
b) a polymerase (e.g., Klenow (exo⁻)); and
c) a target nucleic acid.

Such compositions may also contain water or an aqueous buffer and other reagents (e.g., the polymerase buffer components and appropriate primers as described in the Examples). Generally, in live scrubbing, the labeled nucleotide(s) is/are in molar excess over their respective scrubbing oligonucleotides and/or the nucleotide impurities are kinetically favored over the labeled nucleotide(s) for incorporation in the scrubbers. For example, the labeled nucleotide(s) may be in at least a 2-, 5-, 10-, 50-, 100-, or 1000-molar excess over their respective scrubbing oligonucleotides. The optimal concentration of a scrubbing nucleotide is determined for each specific system. In the illustrative embodiments, the scrubbing oligonucleotides are at the final concentration of about 20 μM, however, it can range, for example, from 100 nM to 500 μM.

In some embodiments, only one species of labeled nucleotide is present in the composition along with any one, any two, any three, or all four types of scrubbing oligonucleotides (A-scrubber, T/U-scrubber, G-scrubber, and C-scrubber). In some embodiments, at least one of the scrubbing oligonucleotides present in the composition removes the nucleotide impurities of the same species as the labeled nucleotide(s) in the same composition.

The invention further provides methods of sequencing a target nucleic acid by synthesis that utilize “live” scrubbing. Sequencing by synthesis is described in detail below. Generally, in such methods, a target nucleic acid is exposed to a) polymerase, b) at least one type of a labeled nucleotide (e.g., A, G, T, U, or C), and c) a scrubbing oligonucleotide, under conditions that allow the polymerase to incorporate the labeled nucleotide(s) into the chain complementary to the target nucleic acid. This step may be practiced with a sequential addition of a single nucleotide species, followed by detection/imaging, which is then followed by the next cycle. Alternatively, the step may be practiced with a simultaneous addition of multiple nucleotide species. In some such embodiments, four types of nucleotides, each labeled with a different color-coded label are added simultaneously and the incorporation or nucleotides is detected in real time.

The sequence of the target nucleic acid is determined based upon the order of incorporation of the labeled nucleotide(s) into the complementary chain. In the illustrative embodiments, the sequencing is performed at a single molecule level, as described more specifically below. In some such embodiments, multiple target nucleic acid molecules are directly or indirectly attached to a support and are individually optically resolvable. In yet other embodiments, the polymerase may be directly or indirectly attached to a support.

### Sequencing Platforms

The invention can be used on any suitable sequencing-by-synthesis platform. As described above, four major sequencing-by-synthesis platforms are currently available: the Genome Sequencers from Roche/454 Life Sciences, the 1G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnolgies. Each of these platforms can be used in the methods of the invention. In some embodiments, the sequencing platforms used in the methods of the present invention have one or more of the following features:
1) four differently optically labeled nucleotides are utilized (e.g., 1G Analyzer, Pacific BioSciences, and Visigen);
2) sequencing-by-ligation is utilized (e.g., SOLiD);
3) pyrophosphate detection is utilized (e.g., Roche/454);
4) four identically optically labeled nucleotides are utilized (e.g., Helicos);
5) fluorescent energy transfer (FRET) is utilized (e.g., Visigen).

In some embodiments, a plurality of nucleic acid molecules being sequenced is bound to a support. To immobilize the nucleic acid on a support, a capture sequence/universal priming site can be added at the 3′ and/or 5′ end of the template. The nucleic acids may be bound to the solid support by hybridizing the capture sequence to a complementary sequence covalently attached to the solid support. The capture sequence (also referred to as a universal capture sequence) is a nucleic acid sequence complimentary to a sequence attached to a solid support that may dually serve as a universal primer. In some embodiments, the capture sequence is polyNₙ, wherein N is U, A, T, G, or C, n≧5, e.g., 20-70, 40-60, e.g., about 50. For example, the capture sequence could be polyT₄₀₋₅₀ or its complement.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair.

The solid support may be, for example, a glass surface such as described in, e.g., U.S. Patent App. Pub. No. 2007/0070349. The surface may be coated with an epoxide, polyelectrolyte multilayer, or other coating suitable to bind nucleic acids. In preferred embodiments, the surface is coated with epoxide and a complement of the capture sequence is attached via an amine linkage. The surface may be derivatized with avidin or streptavidin, which can be used to attach to a biotin-bearing target nucleic acid. Alternatively, other coupling pairs, such as antigen/antibody or receptor/ligand pairs, may be used. The surface may be passivated in order to reduce background. Passivation of the epoxide surface can be accomplished by exposing the surface to a molecule that attaches to the open epoxide ring, e.g., amines, phosphates, and detergents.

Subsequent to the capture, the sequence may be analyzed, for example, by single molecule detection/sequencing, e.g., as described in the Example and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide may be incorporated and multiple lasers may be utilized for stimulation of incorporated nucleotides.

### Target Nucleic Acids

The length of the target nucleic acid may vary. The average length of the target nucleic acid may be, for example, at least 300, 350, 400, 450, 500, 550, 600, 700, 800, 900, 1000, 2000, 3000, 4000, 5000 nts or longer. In some embodiments, the length of the target is between 300 and 5000 nts, 400 and 4000 nts, or 500 and 3000 nts.

Target nucleic acids can come from a variety of sources. For example, nucleic acids can be naturally occurring DNA or RNA (e.g., mRNA or non-coding RNA) isolated from any source, recombinant molecules, cDNA, or synthetic analogs. For example, the target nucleic acid may include whole genes, gene fragments, exons, introns, regulatory elements (such as promoters, enhancers, initiation and termination regions, expression regulatory factors, expression controls, and other control regions), DNA comprising one or more single-nucleotide polymorphisms (SNPs), allelic variants, and other mutations. The target nucleic acid may also be tRNA, rRNA, ribozymes, splice variants, or antisense RNA.

Target nucleic acids may be obtained from whole organisms, organs, tissues, or cells from different stages of development, differentiation, or disease state, and from different species (human and non-human, including bacteria and virus). Various methods for extraction of nucleic acids from biological samples are known (see, e.g., Nucleic Acids Isolation Methods, Bowein (ed.), American Scientific Publishers, 2002). Typically, genomic DNA is obtained from nuclear extracts that are subjected to mechanical shearing to generate random long fragments. For example, genomic DNA may be extracted from tissue or cells using a Qiagen DNeasy Blood & Tissue Kit following the manufacturer's protocols.

Other details and variations of the sequencing methods are provided below.

### Other General Considerations

A. Nucleotides—Nucleotides useful in the invention include any nucleotide or nucleotide analog, whether naturally occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, including chain-terminating analogs.

Nucleotides for nucleic acid sequencing, according to the invention, preferably comprise a detectable label that is directly or indirectly detectable. Preferred labels include optically detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron® Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Additional fluorescent dyes that can be used in the methods of the invention include ATTO dyes (such as, e.g., ATTO 390, 425, 465, 488, 495, 520, 532, 550, 565, 590, 594, 610, 611X, 620, 633, 635, 637, 647, 647N, 655, 680, 700, 725, and 740) available from Atto Technologies (Germany).

Labels other than fluorescent labels are contemplated, including other optically detectable labels. In some embodiments, a labeled nucleotide comprises a fluorescent label attached to the nitrogenous base, optionally, via a disulfide, such as illustrated by Formula I and Formula II below.

B. Nucleic Acid Polymerases—Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg et al. (1991) Gene, 108:1, Stratagene), Pyrococcus woesei (Pwo) DNA polymerase (Hinnisdaels et al. (1996), Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh et al. (1977) Biochim. Biophys. Acta, 475:32), Thermococcus litoralis (Tli) DNA polymerase (also referred to as Vent® DNA polymerase, Cariello et al. (1991) Polynucleotides Res., 19:4193; New England Biolabs), 9° Nm® DNA polymerase (New England Biolabs), Stoffel fragment, ThermoSequenase® (Amersham Pharmacia Biotech UK), Therminator® (New England Biolabs), Thermotoga maritima (Tma) DNA polymerase (Diaz et al. (1998) Braz. J. Med. Res., 31:1239), Thermus aquaticus (Taq) DNA polymerase (Chien et al. (1976) J. Bacteoriol., 127:1550), DNA polymerase, Pyrococcus kodakaraensis KOD DNA polymerase (Takagi et al. (1997) Appl. Environ. Microbiol., 63:4504), JDF-3 DNA polymerase (from thermococcus sp. JDF-3, PCT Patent Application Publication WO 01/32887), Pyrococcus GB-D (PGB-D) DNA polymerase (also referred as Deep Vent® DNA polymerase, Juncosa-Ginesta et al. (1994) Biotechniques, 16:820; New England Biolabs), UITma DNA polymerase (from thermophile Thermotoga maritima; Diaz et al. (1998) Braz. J. Med. Res., 31:1239; PE Applied Biosystems), Tgo DNA polymerase (from thermococcus gorgonarius, Roche Molecular Biochemicals), E. coli DNA polymerase I (Lecomte et al. (1983) Polynucleotides Res., 11:7505), T7 DNA polymerase (Nordstrom et al. (1981) J. Biol. Chem., 256:3112), and archaeal DP11/DP2 DNA polymerase II (Cann et al. (1998) Proc. Natl. Acad. Sci. USA, 95:14250-5).

While thermophilic polymerases are contemplated by the invention, preferred polymerases are mesophilic. Mesophilic DNA polymerases include, but are not limited to, E. coli DNA polymerase I and Klenow (exo⁻) fragment. Polymerases, irrespective of source, are preferably exonuclease-deficient in many implementations.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin (1997) Cell, 88:5-8; Verma (1977) Biochim. Biophys. Acta, 473:1-38; Wu et al. (1975) CRC Crit. Rev. Biochem., 3:289-347).

C. Surfaces/Solid support—In a preferred embodiment, nucleic acid template molecules are attached to a solid support (“substrate”). Substrates for use in the invention can be two-or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate is coated to allow optimum optical processing and nucleic acid attachment. Substrates for use in the invention can also be treated to reduce background. Exemplary coatings include epoxides, and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Erie Glass (Portsmouth, N.H.). In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate or, at least, do not substantially impair template binding.

Various methods can be used to anchor or immobilize the primer to the surface of the substrate. The immobilization can be achieved through direct or indirect bonding to the surface. The bonding can be by covalent linkage. See, Joos et al. (1997) Analytical Biochemistry, 247:96-101; Oroskar et al. (1996) Clin. Chem., 42:1547-1555; and Khandjian (1986) Mol. Bio. Rep., 11:107-11. A preferred attachment is direct amine bonding of a terminal nucleotide of the template or the primer to an epoxide integrated on the surface. The bonding also can be through non-covalent linkage. For example, biotin-streptavidin (Taylor et al. (1991) J. Phys. D: Appl. Phys., 24:1443) and digoxigenin with anti-digoxigenin (Smith et al. (1992) Science, 253:11220) are common tools for anchoring nucleic acids to surfaces and parallels. Alternatively, the attachment can be achieved by anchoring a hydrophobic chain into a lipid monolayer or bilayer. Other methods known in the art for attaching nucleic acid molecules to substrates can also be used.

D. Detection—Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include the scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity, Mason (ed.), Academic Press, Landon, pp. 1-11 (1993), such as described in Yershov et al. (1996) Proc. Natl. Acad. Sci., 93:4913, or may be imaged by TV monitoring. For radioactive signals, a Phosphorlmager™ device can be used (Johnston et al. (1990) Electrophoresis, 13:566; Drmanac et al. (1992) Electrophoresis, 13:566). Other commercial suppliers of imaging instruments include General Scanning Inc., (Watertown, Mass.; genscan.com), Genix Technologies (Waterloo, Ontario, Canada; confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (“movies”) of fluorophores.

Some embodiments of the present invention use TIRF microscopy for two-dimensional imaging. TIRF microscopy uses totally internally reflected excitation light and is well known in the art. See, e.g., nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently labeled nucleic acid molecules. When a laser beam is totally reflected at the interface between a liquid and a solid substrate (e.g., glass), the excitation light beam penetrates only a short distance into the liquid. The optical field does not end abruptly at the reflective interface, but its intensity falls off exponentially with distance. This surface electromagnetic field, called the “evanescent wave,” can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

The following Example provides illustrative embodiments of the invention and does not in any way limit the invention.

### EXAMPLES

### Example 1

### Labeled Nucleotides

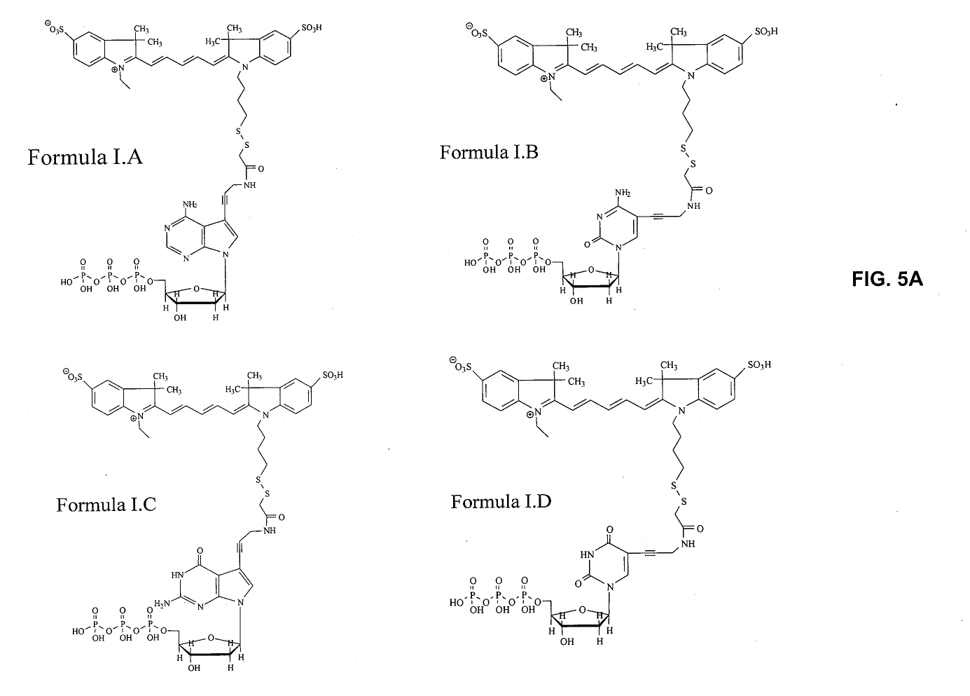

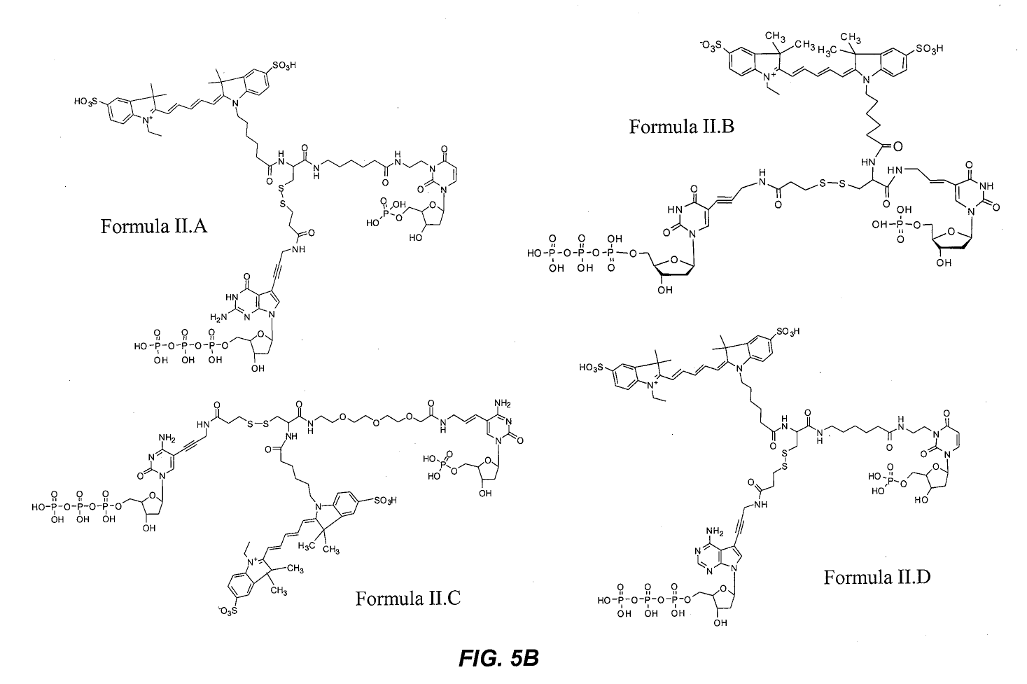

The Cy5 labeled 12SS nucleotides (Formulas I.A-I.D) or tether analogs, also referred to as Virtual Terminators™, (Formulas II.A-II.D) are used, as shown in FIGS. 5A and 5B. The 12SS nucleotides were purchased from PelkinElmer. The following abbreviated nomenclature is used: I.A Cy5-12SS-dATP (7-deaza); I.B Cy5-12SS-dCTP (C-5); I.C.; Cy5-12SS-dGTP (7-deaza); I.D Cy5-12SS-dUTP (C-5); II.A Cy5-G*pU; II.B Cy5-U*pU; II.C Cy5-C*pC; and II.D Cy5-A*pU.

### Example 2

### HPLC Purification of Dye-Labeled Nucleotides

The analogues are stored in 1×TE buffer at 1 mM concentration and at or below −20° C. The HPLC purification system is composed of three major components: a pump system (Waters 1525), a photodiode detector (Waters 2996), and HPLC columns (Luna C18 from Phenomenex). A total of four columns and injection ports are used, one set for each type of dNTP so as to minimize any possible cross-contaminations. The injections are performed according to the user manual (Waters) The exact amount of nucleotide purified at any one time is gauged to the dimensions of the column used, for example 100-200 nmoles injected into columns measuring 3 mm×150 mm. Buffers used for the process may be specially treated by either filtration through a bed of activated charcoal or exposed to metallic substances which have high affinities for thiol compounds, such as gold or silver, or both processes. This treatment is to ensure the lowest levels of contaminating mercaptans which catalyze cleavage of the —SS— in the nucleotide analogs if interest and produce dark nucleotides. The flow rate is 1 ml/min and the concentration gradient is set as follows: 0-2 min, 50 mM TEAB buffer; 2-32 min, 1.3% per minute methanol gradient increase to 40%; 32-42 min, constant 60% TEAB, 40% methanol gradient; 42-47 min, 4% per minute methanol gradient increase to 60%. The 12SS dNTPs are eluted and collected during the 32-35 min period. The purified dNTPs are collected directly into a light protected vessel and exposure to room light minimized. Upon collection, optical density is taken to measure the concentrations, which is usually in the range of 50-100 μM (in 40% methanol). Purified nucleotides are stored in the HPLC elution buffer and used directly to avoid any breakdown resulting from standard practices of roto evaporation to remove solvents. Purified nucleotides are stored in separate aliquots at −60° C.

### Example 3

### Pre-Scrubbing of the Sequencing Reaction Mixture

The following components are mixed to pre-scrub the sequencing reaction mixture of any impurities. The 50 μl reaction contains: 1× polymerase reaction buffer (20 mM Tris base, 10 mM KCl, 10 mM NaCl, 10 mM (NH₄)₂SO₄, 0.1% Triton® X-100) 10 mM MgCl₂, 0.5 μM primer:DNA duplex, 225 nM Klenow (exo−) polymerase (1,000 U/ml), 100 μM HPLC-purified dNTP, and 22.5 μM of each of the four types of hairpin scrubber oligonucleotides (SEQ ID NOs:1-4). The scrubbers are purchased from a vendoe with the specification of the HPLC purification.

The pre-scrubbing reaction is carried out for 5 min at 37° C. and then stopped with 10 μl of 100 mM EDTA. The quenched reaction mix is then added to the upper chamber of μCon-10 column with 190 μl of dH₂O. The column is spun at 14 rcf for 25 min. The filtered volume is adjusted to 250 μl with dH₂O, bringing the final nucleotide concentration to about 20 μM. The final solution is stored at −20° C.

### Example 4

### Preparation of the Polymerase Buffer for Live Scrubbing

Hairpin scrubbers, described in Example 2, are used. Scrubbing solutions are prepared as follows.

First, an aliquot of 117.5 μl of each of the four 100 μM scrubber solutions is taken and placed into four 200 μl PCR tubes. Using a thermocycler, each tube is heated to 95° C. for 5 min, thereupon the temperature is lowered to 50° C. over 30 sec and held at 50° C. for 10 min. The vials are then removed from the thermocycler and allowed to cool to room temperature.

Second, to prepare the polymerase buffer the following components are added to an appropriately sized disposable plastic tube: 1.21 g of Tris base, 0.37 g of KCl, 0.29 g of NaCl, 0.66 g of (NH₄)₂SO₄, 0.5 ml of Triton® X-100, and 40 ml of MilliQ water. The solution is titrated to pH 8.8, using 1N HCl. The final volume should be around 50 ml.

Third, the polymerase buffer solution (50 ml) is mixed with 112.5 μl of each type of scrubber prepared in the first step. The solution is then filtered through 0.22 μm filter. The final solution is packaged into either 2 or 8 ml tubes and stored at −20° C. The scrubber solution is used for sequencing in the same way the polymerase reaction buffer lacking the scrubbers is used.

### Example 5

### Single Molecule Sequencing

Epoxide-coated glass slides are prepared for oligo attachment. Epoxide-functionalized 40 mm diameter #1.5 glass cover slips (slides) are obtained from Erie Scientific (Salem, N.H.). The slides are preconditioned by soaking in 3×SSC for 15 minutes at 37° C. Next, a 500-pM aliquot of 5′ aminated oligonucleotide (SEQ ID NO:5) is incubated with each slide for 30 minutes at room temperature in a volume of 80 ml. The slides are then treated with phosphate (1 M) for 4 hours at room temperature in order to passivate the surface. Slides are then stored in 20 mM Tris, 100 mM NaCl, 0.001% Triton® X-100, pH 8.0 at 4° C. until they are used for sequencing.

For sequencing, the slide is placed in a modified FCS2 flow cell (Bioptechs, Butler, Pa.) using a 50-μm thick gasket. The flow cell is placed on a movable stage that is part of a high-efficiency fluorescence imaging system built based on a Nikon TE-2000 inverted microscope equipped with a total internal reflection (TIR) objective. The slide is then rinsed with HEPES buffer with 100 mM NaCl and equilibrated to a temperature of 50° C. An aliquot of the synthetic oligonucleotides (SEQ ID NOs:6-15) labeled with Cy3 at the 5′ end were diluted in 3×SSC to a final concentration of 200 pM (each). A 100-μl aliquot is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains the ten oligonucleotides/primer template duplex randomly bound to the glass surface. The temperature of the flow cell is then reduced to 37° C. for sequencing and the objective is brought into contact with the flow cell.

Further, cytosine triphosphate, guanidine triphosphate, adenine triphosphate, and uracil triphosphate, each having a cleavable cyanine-5 label (at the 7-deaza position for ATP and GTP and at the C5 position for CTP and UTP (PerkinElmer) are stored separately in the buffer containing 20 mM Tris-HCl, pH 8.8, 50 μM MnSO₄, 10 mM (NH₄)₂SO₄, 10 mM HCl, and 0.1% Triton X-100, and 50 U Klenow exo⁻ polymerase (NEB).

Note on the nucleotides: The nucleotides are all HPLC purified. For the pre-scrubbing experiment, the HPLC purified and pre-scrubbed nucleotides were used. For the in situ scrubbing experiment, the HPLC purified nucleotides were used, but the buffer contains 22.5 nM of each type of hairpin scrubber, per protocols substantially as described in the prior Examples.

Sequencing proceeds as follows. First, initial imaging is used to determine the positions of duplex on the epoxide surface. The Cy3 label attached to the synthetic oligo fragments is imaged by excitation using a laser tuned to 532 nm radiation (Verdi V-2 Laser, Coherent, Santa Clara, Calif.) in order to establish duplex position. For each slide only single fluorescent molecules that are imaged in this step are counted. Imaging of incorporated nucleotides as described below is accomplished by excitation of a cyanine-5 dye using a 635-nm radiation laser (Coherent). 100 nM Cy5-CTP is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 μl volumes each, followed by 150 mM HEPES/150 mM NaCl/pH 7.0 (“HEPES/NaCl”) (10 times at 60 μl volumes). An oxygen scavenger containing 30% acetonitrile and scavenger buffer (134 μl 150 mM HEPES/100 mMNaCl, 24 μl 100 mM Trolox in 150 mM MES, pH 6.1, 10 μl 100 mM DABCO in 150 mM MES, pH 6.1, 8 μl 2M glucose, 20 μl 50 mM NaI, and 4 μl glucose oxidase (USB) is next added. The slide is then imaged (100 frames) for 2 seconds using an Inova 301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 μl) and HEPES/NaCl (60 μl). Next, the cyanine-5 label is cleaved off incorporated CTP by introduction into the flow cell of 50 mM TCEP/250 mM Tris, pH 7.6/100 mM NaCl for 5 minutes, after which the flow cell is rinsed 5 times each with SSC/HEPES/SDS (60 μl) and HEPES/NaCl (60 μl). The remaining nucleotide is capped with 50 mM iodoacetamide/100 mM Tris, pH 9.0/100 mM NaCl for 5 minutes followed by rinsing 5 times each with SSC/HEPES/SDS (60 μl) and HEPES/NaCl (60 μl). The scavenger is applied again in the manner described above, and the slide is again imaged to determine the effectiveness of the cleave/cap steps and to identify non-incorporated fluorescent objects.

The procedure described above is then conducted 100 nM Cy5-dATP, followed by 100 nM Cy5-dGTP, and finally 100 nM Cy5-dUTP. Uridine may be used instead of Thymidine due to the fact that the Cy5 label is incorporated at the position normally occupied by the methyl group in Thymidine triphosphate, thus turning the dTTP into dUTP. The procedure (expose to nucleotide, polymerase, rinse, scavenger, image, rinse, cleave, rinse, cap, rinse, scavenger, final image) is repeated for a total of 48 cycles.

Once the desired number of cycles is completed, the image stack data (i.e., the single-molecule sequences obtained from the various surface-bound duplexes) are aligned to the reference barcode sequences. The individual single molecule sequence read lengths obtained range from 2 to 16 consecutive nucleotides with about 12.6 consecutive nucleotides being the average length and only those greater than 9 bases in length with less than 2 errors where used in the final analysis.

Once the desired number of cycles is completed, the image stack data (i.e., the single-molecule sequences obtained from the various surface-bound duplex) are aligned to the reference sequence. Only the individual single molecule sequence read lengths obtained ranging from 6 and above are analyzed. A missing base error is detected, when the single molecule sequence contains a gap (of one or more nucleotides) compared to the reference sequence.

### Example 6

### Comparison of Pre-Scrubbing and Live Scrubbing

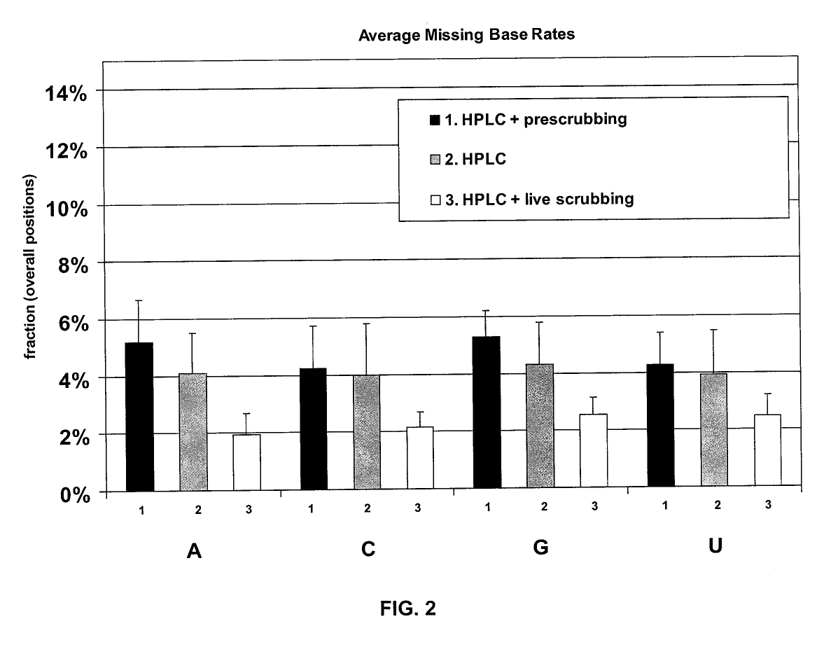

A study comparing single molecule sequencing reaction under different scrubbing conditions was performed substantially as described in Examples 1-5. The following conditions were compared: 1) nucleotides were purified by HPLC and pre-scubbed; 2) nucleotides were purified by HPLC only; and 3) nucleotides were purified by HPLC, and live scrubbing was performed during the sequencing reaction. The missing base error rates were evaluated, and the results are presented in FIG. 2. The results suggest that live scrubbing is more effective in reducing the error rate than pre-scrubbing. Pre-scrubbing in combination with the HPLC purification did not appear to provide any additional benefits over HPLC alone, suggesting that the contaminating nucleotide degradation may occur primarily in the course of the sequencing reaction.

### Example 7

### Comparison of Single-Site Pre-Scrubbing and Live Scrubbing

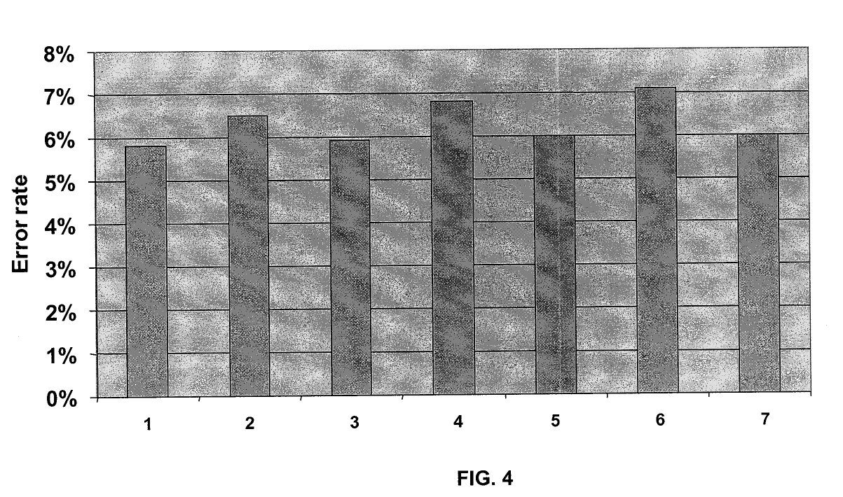

A study comparing live scrubbing with single-site hairpin scrubbers vs. homopolymeric hairpin was performed substantially as described in Examples 1-5, using the scrubbers as set out in FIG. 3B. Sequencing was performed substantially as described in Example 3, except the nucleotides were the tether analogs as described in U.S. Pat. App. Pub. No. 2007/0190546. The tether analogs were used at the following concentrations: 250 nM dCTP and dUTP, 500 nM dATP and dGTP. The incubations were conducted for 4 min at 37° C. The MnSO₄ was used at a final concentration of 75 μM. The total error rate, which includes the missing base rates (discussed above), insertion rate (when there is a gap in the reference compared to the single molecule sequence), and substitution rate (a base in the reference was substituted for a different base in the single molecule sequence), was evaluated. FIG. 4 shows results of several studies comparing the total error rates (missing bases, insertions, and substitutions) in sequencing by synthesis, utilizing single-site scrubbers (bars 1-6) or homopolymeric scrubbers (bar 7). The average error rates for all four bases are reported (average of A, C, G, U). The results suggest that, for the tether analogs, live scrubbing with the homopolymeric scrubbers has similar error rates as the single-site scrubbers.

All publications, patents, patent applications, and biological sequences cited in this disclosure are incorporated by reference in their entirety.

1. A method of sequencing a target nucleic acid, the method comprising:
a) exposing a target nucleic acid to a reaction mixture comprising:
i) a polymerase;
ii) a labeled nucleotide; and
iii) a scrubbing oligonucleotide comprising a double-stranded region and a single-stranded overhang, said scrubbing oligonucleotide capable of incorporating a contaminant nucleotide;
b) allowing the polymerase to incorporate the labeled nucleotide into a chain complementary to the target nucleic acid;
c) optionally, repeating steps a) and b) with a labeled nucleotide of the same or different species; and
d) determining the sequence of the target nucleic acid based upon the order of incorporation of the labeled nucleotide(s) into the complementary chain.

2. The method of claim 1, wherein the target nucleic acid and/or the polymerase is/are attached to a support.

3. The method of claim 2, wherein the target nucleic acid is individually optically resolvable.

4. The method of claim 1, wherein the scrubbing oligonucleotide is in solution.

5. The method of claim 1, wherein the single-stranded overhang in the scrubbing oligonucleotide is 1-20 nucleotides long.

6. The method of claim 1, wherein the site of incorporation at a 3′ end of the scrubbing oligonucleotide is complementary to the labeled nucleotide species.

7. The method of claim 1, wherein the scrubbing oligonucleotide has a hairpin structure.

8. The method of claim 1, wherein the scrubbing oligonucleotide comprises a homopolymeric sequence at the site of incorporation at a 3′ end.

9. The method of claim 8, wherein the homopolymeric sequence is 7 nucleotides long or shorter.

10. A composition comprising:
1) water or an aqueous buffer; and
2) at least one type of a scrubbing oligonucleotide in solution; and
3) one or both of a) and b):
a) at least one type of labeled nucleotide species chosen from A, G, T, U, or C; and
b) a polymerase,
wherein the scrubbing oligonucleotide comprises a double-stranded region, an optional loop region, and a single-stranded 5′ overhang region that allows for incorporation of one or more complementary nucleotides at a 3′ end of the double-stranded region.

11. The composition of claim 10, wherein the composition comprises b) the polymerase.

12. The composition of claim 10, wherein the composition comprises at least one type of labeled nucleotide species chosen from A, G, T, U, or C.

13. The composition of claim 10, wherein the composition comprises both a) and b).

14. The composition of claim 10, wherein each of the scrubbing oligonucleotides comprises a loop region.

15. The composition of claim 10, wherein the composition comprises four types of scrubbing oligonucleotides, each type capable of incorporating, at the first position at a 3′ end, differing nucleotide species chosen from A, G, T, U, or C.

16. The composition of claim 10, wherein the scrubbing oligonucleotides comprise a homopolymeric sequence at the site of incorporation at the 3′ end.

17. The composition of claim 10, wherein the at least one scrubbing oligonucleotide has a melting temperature of above 65° C. and/or a GC content of the double-stranded region of above 40%.

18. The composition of claim 10, wherein the at least one scrubbing oligonucleotide comprises a structure as set out in FIG. 3A.

19. The composition of claim 12, wherein the at least one scrubbing oligonucleotide is capable of incorporating, at the first position at a 3′ end, a nucleotide species of the same kind as that of the labeled nucleotide.

20. The composition of claim 12, wherein the labeled nucleotide is a deoxyribonucleotide.

21. The composition of claim 12, wherein the labeled nucleotide(s) is/are in at least a two-fold molar excess over the scrubbing oligonucleotide(s).

22. The composition of claim 12, wherein the at least one labeled nucleotide comprises a fluorescent label.

23. A method of reducing unlabeled nucleotide impurities during a nucleic acid polymerization reaction with labeled nucleotides, the method comprising:
a) contacting a target nucleic acid with the composition of claim 12;
b) allowing an unlabeled nucleotide impurity to incorporate into the at least one scrubbing nucleotide; and
c) allowing the labeled nucleotide to incorporate into the target nucleic acid.

24. The method of claim 23, further comprising pre-purifying the labeled nucleotide prior to step a).

25. The method of claim 23, wherein incorporation of the unlabeled nucleotide impurity into the scrubbing oligonucleotide is kinetically favored over the labeled nucleotide.


SEQUENCE LISTING (16 sequences)

SEQ ID NO: 1 - DNA, 60 bp, Artificial sequence
  Synthetic oligonucleotide
aatgtcacgtgagctgactgtatgagtagtaccgacatctactactcatacagtcagctc

SEQ ID NO: 2 - DNA, 60 bp, Artificial sequence
  Synthetic oligonucleotide
aatgtcacgggagctgactgtatgagtagtaccgacatctactactcatacagtcagctc

SEQ ID NO: 3 - DNA, 60 bp, Artificial sequence
  Synthetic oligonucleotides
aatgtcacgcgagctgactgtatgagtagtaccgacatctactactcatacagtcagctc

SEQ ID NO: 4 - DNA, 60 bp, Artificial sequence
  Synthetic oligonucleotide
aatgtcacgagagctgactgtatgagtagtaccgacatctactactcatacagtcagctc

SEQ ID NO: 5 - DNA, 32 bp, Artificial sequence
  Synthetic oligonucleotide
tccacttatccttgcatccatcctctgccctg

SEQ ID NO: 6 - DNA, 57 bp, Artificial sequecne
  Synthetic oligonucleotide
cacagtctcagctatgtagcgatacagggcagaggatggatgcaaaggataagtgga

SEQ ID NO: 7 - DNA, 57 bp, Artificial sequence
  Synthetic oligonucleotide
cactagtatgtctagcgcgtagcacagggcagaggatggatgcaaaggataagtgga

SEQ ID NO: 8 - DNA, 57 bp, Artificial sequence
  Synthetic oligonucleotide
cagtcgtcatactactagagcgatcagggcagaggatggatgcaaaggataagtgga

SEQ ID NO: 9 - DNA, 57 bp, Artificial sequence
  Synthetic oligonucleotide
cactatagtacgtagtatgatcgtcagggcagaggatggatgcaaaggataagtgga

SEQ ID NO: 10 - DNA, 57 bp, Artifical Sequence
catacgatgcagtacacatagtctcagggcagaggatggatgcaaaggataagtgga

SEQ ID NO: 11 - DNA, 57 bp, Artificial sequence
  Synthetic oligonucleotide
catatagagtctcacgtcatgcagcagggcagaggatggatgcaaaggataagtgga

SEQ ID NO: 12 - DNA, 57 bp, Artificial sequence
  Synthetic oligonucleotide
catcatactcgtgagcgtagtatacagggcagaggatggatgcaaaggataagtgga

SEQ ID NO: 13 - DNA, 56 bp, Artificial sequence
  Synthetic oligonucleotide
cagctcagcgatgactgtcgataccagggcagaggatggatgcaaaggataagtgg

SEQ ID NO: 14 - DNA, 57 bp, Artificial sequence
  Synthetic oligonucleotide
cagcgcatagcatacatagatgtccagggcagaggatggatgcaaaggataagtgga

SEQ ID NO: 15 - DNA, 57 bp, Artificial sequence
  Synthetic oligonucleotide
cagcgcacgatatatgctatcagccagggcagaggatggatgcaaaggataagtgga

SEQ ID NO: 16 - DNA, 64 bp, Artificial sequence
  Synthetic oligonucleotide
aatgtcacgtttttgagctgactgtatgagtagtaccgacatctactactcatacagtcagctc